(19) United States
(12) Patent Application Publication
(10) Pub. No.: US 2024/0326102 A1
(43) Pub. Date: Oct. 3, 2024

(54) PROCESS FOR REPROCESSING A UTENSIL FROM BACTERIAL NANOCELLULOSE LOADED WITH AN ADDITIVE

(71) Applicant: HYBRID REALITY GMBH, Berlin (DE)

(72) Inventors: Ani MANERA DA SILVA, Amsterdam (NL); Cord LABUHN, Berlin (DE)

(73) Assignee: HYBRID REALITY GMBH, Berlin (DE)

(21) Appl. No.: 18/573,461

(22) PCT Filed: Jun. 20, 2022
(86) PCT No.: PCT/EP2022/066695

§ 371 Date: Dec. 22, 2023

(30) Foreign Application Priority Data
Jun. 23, 2021 (DE) 10 2021 116 254.8

(51) Int. Cl. B08B 3/08; A61J 11/00; A61L 2/07; A61L 2/08; C08B 15/00; C11D 7/06

(52) U.S. Cl. CPC B08B 3/08; A61J 11/0075; A61L 2/07; A61L 2/087; C08B 15/00; C11D 7/06; A61L 2202/17

(57) ABSTRACT

The invention relates to a process for reprocessing a utensil made of a nonwoven made of bacterial nanocellulose loaded with an additive, comprising the process steps of washing the loaded utensil in an alkaline solution and subsequently rinsing and sterilizing the utensil, a rubber dam made of a dimensionally stable hydrogel made of bacterial nanocellulose and the use of a hydrogel made of bacterial nanocellulose as a rubber dam.

The invention relates to a process for reprocessing a utensil made of bacterial nanocellulose loaded with an additive, comprising the process steps of washing the loaded utensil in an alkaline solution and subsequently rinsing and sterilizing the utensil, a rubber dam made of a hydrogel made of bacterial nanocellulose and the use of a hydrogel made of bacterial nanocellulose as a rubber dam.

Dimensionally stable hydrogels made from bacterial nanocellulose are known. These are produced by cultivating a suitable bacterial strain in an aqueous and acid-buffered nutrient medium, whereby a dimensionally stable hydrogel forms at the interface between the nutrient medium and the air during the cultivation process, which can sometimes last several weeks. Depending on the manufacturing process, hydrogels produced in this way also meet the requirements for vegan products. In the following, these dimensionally stable hydrogels are also referred to as “nanocellulose gels” or “BNC nonwovens”.

Nanocellulose gels can be used as massage sponges, washcloths, wet wipes or protective films due to their structural similarity to human skin, their good compatibility with the human organism and their high water retention capacity. Nanocellulose gels can be loaded and equipped with special properties (such as color, taste, fragrance, surface structure, permeability, active ingredient loading). In-situ modification, i.e. influencing the synthesis during the cultivation process by adding different additives to the culture medium, is also known. It is also known to modify biomaterials based on bacterial nanocellulose after it has been synthesized (post-modification).

‘Microbial polymers’ include polymers produced by a microorganism such as bacteria, fungi or algae. Nanocellulose is preferably synthesized by cultivating microbial strains such as Gluconacetobacter, Enterobacter, Agrobacterium, Pseudomonas, and Rhizpobium. In addition to Gluctonacetobacter hansenii and Gluconacetobacter kombuchae, the bacterial strain Gluconacetobacter xylinus, which has also been most extensively researched and documented in this context, is particularly suitable for the production of nanocellulose gels. The nanocellulose gel is produced by microorganisms at the interface between air and a nutrient medium containing D-glucose. In the alternative production processes discussed below, sucrose in aqueous solution is the carbon source. The bacteria expel the cellulose as fibrils, which assemble into fibers at the interface between the nutrient medium and the air. The result is a three-dimensionally interwoven fiber network consisting of about 99% water and 1% nanocellulose.

Thanks to the special material properties of this biopolymer, its extremely high biocompatibility, structural similarity to the body's own protein-based tissue, variety of shapes and numerous modification options, it is already being used in pharmaceuticals, medicine, cosmetics and food chemistry. Bacterial nanocellulose can be sterilized under normal conditions and is characterized by a high water content and mechanical stability, while its surface and consistency are described as pleasantly soft and particularly smooth. In cosmetics, for example, it appears enriched with active ingredients and vitamins in the form of face masks. In medicine, blood vessels, implants and wound dressings made from bacterial nanocellulose are being researched and used.

### SOURCES

K.-Y. Lee, J. J. Blaker, A. Bismarck: Surface functionalization of bacterial cellulose as the route to produce green polylactide nanocomposites with improved properties, Composites Science and Technology (2009),
D. Klemm, D. Schumann, F. Kramer, N. Heßler, M. Hornung, H.-P. Schmauder, S. Marsch: Nanocelluloses as Innovative Polymers in Research and Application. Advances in Polymer Science (2006), 205(Polysaccharides II).
H. Wang, F. Guan, X. Ma, S. Ren: Production and performance determination of modified bacterial cellulose, Shipin Keji (2009), (5), 28-31;
N. Hessler, D. Klemm: Alteration of bacterial nanocellulose structure by in situ modification using polyethylene glycol and carbohydrate additives, Cellulose (Dordrecht, Netherlands) (2009), 16(5), 899-910;
D. Klemm, D. Schumann, F. Kramer, N. Heßler, M. Hornung, H.-P. Schmauder, S. Marsch: Nanocelluloses as Innovative Polymers in Research and Application. Advances in Polymer Science (2006), 205(Polysaccharides II), 49-96;
M. Seifert: Modification of the structure of bacterial cellulose by the composition of the culture medium in the cultivation of Acetobacter xylinum.

Nanocellulose gels according to the invention in the form of purified nonwovens can be used for massage purposes, are used in physiotherapy, healing practices, osteopathy, body therapy (also as cooling or heating pads, massage aids, haptic stimulants) as well as in body hygiene (wet wipes, refreshing wipes, probiotic washcloths), for disinfection (loaded with disinfectants) as a stimulation aid or protective film and actively for medical purposes. In addition, nanocellulose nonwoven can be used as a massage glove, washcloth or a means of applying cosmetic products (lotions, creams, oils) over large areas. There is an increased demand for nanocellulose gels that can also, but not exclusively, be individually loaded by a user after the gels have been synthesized. An unloaded nanocellulose gel is required for this post-modification. The synthesized hydrogel must be as free as possible from bacterial residues that may adhere during the synthesis of the gel or be retained in the fiber network. Other contaminants, particularly those that are harmful to health, must also be at least below a limit value. At the same time, it must be taken into account during the cleaning process that the hydrogel is an organic material, so the cleaning process must be correspondingly gentle in order to maintain the desired properties.

There are also so-called ‘leak sheets’ (also known in German as Lecktuch or Kofferdam). These are available products made of latex, which prevent sexually transmitted diseases and can be additionally prepared as required with active ingredients (such as commercially available contraceptive gels, spermicides, etc.), dyes, flavors or lubricants. In dentistry, a neutral, sterile rubber dam is used to shield the tooth to be treated from the rest of the oral cavity.

It is therefore the task of the present invention to provide a process for reprocessing a utensil from nanocellulose loaded with an additive, in which the hydrogel is largely free of foreign substances. At the same time, the process should be quick and easy to carry out as well as cost-effective and environmentally friendly. It is also the task of the present invention to provide a rubber dam made of a hydrogel that is more skin-friendly, offers protection and is free of allergens.

The task is solved by means of the process for reprocessing a utensil from bacterial nanocellulose loaded with an additive. Advantageous embodiments of the invention are set out in the sub-claims.

The process for reprocessing a utensil has two process steps: In the first step, the loaded utensil is washed in a caustic solution. Washing with caustic soda solution in a concentration and duration that is adapted to the strength and composition of the hydrogel makes the hydrogel more flexible, softer and more pliable in its cell structure without significantly reducing its stability and water retention capacity for its intended purpose. NaOH can be used in varying concentrations (0.1 molar to 1 molar) to inactivate the bacterial strains and to wash out any entoxins and other foreign substances from the culture medium produced during fermentation. In the second process step, the utensil is sterilized. Sterilization can be used to significantly extend the life cycle of the resistant hydrogels in their final product form and prepare them for reuse. Active ingredient-laden or contaminated hydrogels can thus be prepared for reuse on the skin and can be reused many times if used properly. This way, deposits and unwanted bacteria can be washed out to ensure that the hydrogel is free of germs and impurities before reuse. The pH value of a hydrogel can thus be influenced and adjusted to a desirable range for the skin. In the context of this paper, dimensionally stable hydrogels made of bacterial nanocellulose are also referred to as “nanocellulose gels” or “BNC nonwovens” and are used synonymously.

In a further embodiment of the invention, the caustic solution is a 5 wt % to 50 wt % caustic soda solution. The hydrogel is washed in an alkaline solution containing 5 wt. % to 50 wt. % alkaline solution for a period of 5 min to 400 min at a temperature of 37° C. to 142° C. Preferably, the temperature interval for the cleaning process is 90° C. to 142° C., particularly preferably 100° C. to 142° C. The duration of the washing process is preferably 60 min to 400 min, particularly preferably 120 min to 400 min. The hydrogels produced by this process according to the invention are very easy to wash off due to their surface structure and, particularly in their synthesized pure form, are insensitive to cleaning agents which are also used on the skin (soap, washing-up liquid, etc.). The hydrogels can be boiled in water or sterilized with hot steam and can also be cleaned in the dishwasher without deformation. They are therefore reusable and the hydrogels are completely biodegradable when disposed of properly.

In a further embodiment of the invention, the caustic solution is a 5 wt % to 50 wt % sodium hydroxide solution. Caustic soda, i.e. a solution of NaOH (sodium hydroxide) in water, is a standard material in the chemical industry and is therefore available and inexpensive. NaOH is solid in its pure state and is therefore easy to transport. Disposal is also easy by neutralization with acids or sufficiently strong dilution. Washing with sodium hydroxide solution in a concentration and duration adapted to the strength and composition of the hydrogel makes the hydrogel more flexible, softer and more pliable in its cell structure without significantly reducing its stability and water retention capacity for its intended purpose.

In a further embodiment of the invention, a relative movement is generated between the washing solution and the hydrogel during the washing process. Due to the relative movement, contaminants adhering to the hydrogel and embedded in the hydrogel are removed faster and more thoroughly than in a static cleaning process.

In a further embodiment of the invention, the washing process is carried out in two stages. The two stages of the washing process differ in particular with regard to the concentration of the washing solution and the temperature of the washing solution and the duration of the washing process, or at least in one of the said parameters. In the first stage (coarse cleaning), a 40% to 50% by weight caustic soda solution is used at a temperature at or up to 15° C. below the boiling point of the caustic soda solution used. The boiling point of a 45% by weight caustic soda solution is 142° C. The duration of the first stage of the cleaning process is between 1 min and 150 min, preferably between 100 and 140 min. In the second stage, a washing solution with a lower concentration, lower temperature and longer duration is used. The concentration of the caustic soda is in the range of 0.4% to 8% by weight, the temperature is between 37° C. and 100° C. and thus below the boiling point of the washing solution used. The duration is 1 h to 400 min.

In a further embodiment of the invention, the washing solution is renewed between the first stage and the second stage of the washing process. If the washing process is carried out with the same washing solution, it is useful to renew the solution in order to reduce the concentration of undesired foreign substances.

In an advantageous embodiment of the invention, the washing process is followed by rinsing with distilled water for 30-120 minutes at 80° C., if necessary several times, and sterilization (of the hydrogel). The cleaning process can optionally be completed with sterilization in order to kill other microorganisms and ensure the longest possible shelf life in the packaging. Sterilization can be carried out using hot steam for 20 minutes at 121° C. in an autoclave or in a pressure cooker, for example.

In a further development of the invention, sterilization is carried out using an electron beam (e-beam). Electron beam sterilization is based on the penetration of the products to be sterilized, including the packaging, by high-energy electrons, which have an ionizing and thus germ-killing effect. The sterilization of medical articles by e-beam is one of the most efficient and safest processes, as not only all forms of bacteria, but also viruses, spores and NDA fragments are rendered biologically ineffective.

In a further embodiment of the invention, the utensil is only partially made from a nanocellulose loaded with an additive. The method according to the invention is therefore also suitable for cleaning and thus extending the life cycle of utensils made only partially from a hydrogel.

The task is also solved by means of a rubber dam made of a hydrogel. Further advantageous embodiments of the invention are also set out in the subclaims.

The rubber dam according to the invention is made from a hydrogel of bacterial nanocellulose. In contrast to the available products made of latex, this is a 0.1-3 mm thick protective skin made of a water-based hydrogel according to the invention with natural gliding properties, which prevents viral or bacterial infections or other sexually transmitted diseases and can be additionally prepared as required with active ingredients (such as commercially available contraceptive gels, spermicides, etc.), dyes, coloring foods and flavors. This step can be carried out either in industrial production before packaging for retail or by the consumer.

The hydrogel of bacterial nanocellulose has a fiber length of 5 μm to 15 mm, preferably the nonwoven has a fiber length of 10 μm to 10 mm, more preferably 50 μm to 5 mm and most preferably 100 μm to 1 mm. The diameter of the fibers is 60±15 nm. The density is 1-1.5 g/cm³. The tensile strength of the fibers is 250-400 MPa, preferably 300-350 MPa and particularly preferably 320-335 MPa.

In addition to preventing viral or bacterial infections, the hydrogel can also intensify haptic and visual perception, be used as a toy, haptic stimulant and lubricant or provide protection against mechanical overstress. Another example of protection against overstressing sensitive areas of skin is the use of nipple protectors for competitive sports to reduce friction of the skin against clothing or—with partial perforation to care for the baby—for breastfeeding mothers.

Already in use as a carrier system in cosmetics (active ingredient-soaked cloth masks, face pads) and in medicine (wound dressings, wound plasters) as disposable products in mostly aluminium packaging, the novel applications of bacterial nanocellulose are now aimed at reusability, the reduction of packaging waste and transport routes, customizable application scenarios (DIY cosmetics) and household (hygiene, wet wipes, disinfectant wipes, medical self-care). The use of non-allergenic, organic, biodegradable polymers as an alternative to certain latex or petroleum-based products can be made resource-saving and climate-neutral with this technology and offers the greatest possible compatibility for allergy sufferers and sensitive skin types.

In a further development of the invention, the rubber dam has a cellulose content of more than 0.5% by weight. In all applications, the high water content is of particular importance and the nanocellulose is naturally only reusable for these purposes if it remains hydrated and does not dry out without appropriate preparation. Therefore, storage, loading and renovation take place in liquid, preferably in water or in an aqueous solution.

In a further embodiment of the invention, the rubber dam is loaded with water and a further substance. The loadings are intentional components and are applied to and introduced into the nonwoven during or after the synthesis process of the nonwoven. In cosmetics, for example, such nonwovens appear enriched with active ingredients and vitamins in the form of skin pads and active ingredient carriers. In a further embodiment of the invention, the impurities contain one or more substances from the group of acids mentioned above, trace elements, yeasts, vitamins, as well as organic or inorganic coloring particles, probiotics, antimycotics, disinfectants, alcohol, aloe vera, hyaluronic acid, essential oils, extracts from leaves, roots and fruits, as well as skin particles or body fluids (after use on the skin).

In a further development of the invention, the further substance is a pharmaceutical. In addition to a loading with active ingredients (such as aloe vera, hyaluronic acid, black tea), an acidic loading with probiotic bacterial cultures may be desirable and give the hydrogel special properties, for example in the treatment of certain fungal diseases (with lactic acid and lactic acid bacteria), psoriasis or as a carrier of a specific bacterial culture for the cultivation of bacterial nanocellulose.

Depending on the bacterial strain, culture medium, cultivation temperature and duration as well as other influences, the rubber dam also contains different proportions of the corresponding nutrient solution, which can range from various acids and chemical additives to yeasts, vitamins or organic residues (e.g. from plants such as tea, flowers, fruits or coconut). In certain cases, such as an active, acidic-probiotic charge of the nonwoven (see Kombucha culture) or effective plant components (comparable to CBD from cannabis in-situ instead of in post-modification), this may be desirable.

In a further embodiment of the invention, the rubber dam has a water content of between 20% and 99% by weight. The high water content ensures that the rubber dam remains reusable.

In a further embodiment of the invention, the rubber dam has a thickness of between 0.1 and 3 mm. This results in exceptional material properties such as high chemical and thermal stability, biocompatibility and high mechanical stability and tensile strength in the never-dried state.

In a further embodiment of the invention, the rubber dam is reprocessed according to one or more of claims 1 to 9. In a further embodiment of the invention, the impurities comprise biological impurities and/or chemical impurities. The use of gram-negative bacterial strains during the synthesis of the nonwoven entails the risk of endotoxin contamination of the end products, i.e. the retention of decay products from the outer cell membrane of bacteria, which can trigger undesirable reactions in the human organism. These are only present in harmless concentrations in the rubber dam according to the invention.

The task is also solved by using a hydrogel made of bacterial nanocellulose as a rubber dam.

According to the invention, a hydrogel made of bacterial nanocellulose is used as a rubber dam. The aim of using biocompatible hydrogels in the form of certain products-preferably made from bacterially synthesized nanocellulose (BNC)—is to hydrate or lubricate the skin, to cool or warm certain parts of the body, to supply the skin with active ingredients and to protect the skin from unwanted contact, for example with bacteria, viruses, pollutants, microplastics or dermatologically questionable materials. Objects and surfaces can also be gently cleaned with an appropriately prepared wet wipe (loaded with disinfectant, for example).

In dentistry, for example, a rubber dam is used to shield the tooth to be treated from the rest of the oral cavity, particularly during root canal treatment, a plastic filling, a ceramic inlay filling or amalgam removal. This prevents the inflow of saliva. In addition to shielding the oral cavity for easier saliva-free treatment of the opened tooth, it also prevents foreign bodies from being swallowed or inhaled.

Another application is a nipple protector made from a hydrogel of bacterial nanocellulose. Nipple protectors of this type are used, for example, to protect sensitive areas of skin from overstressing, e.g. in competitive sports to reduce friction of the skin against clothing or—with a partial perforation to care for the baby—for breastfeeding mothers.

Examples of embodiments of the process according to the invention for reprocessing a utensil from bacterial nanocellulose loaded with an additive and of the rubber dam according to the invention are shown in simplified form in the drawings and are explained in more detail in the following description.

It shows:

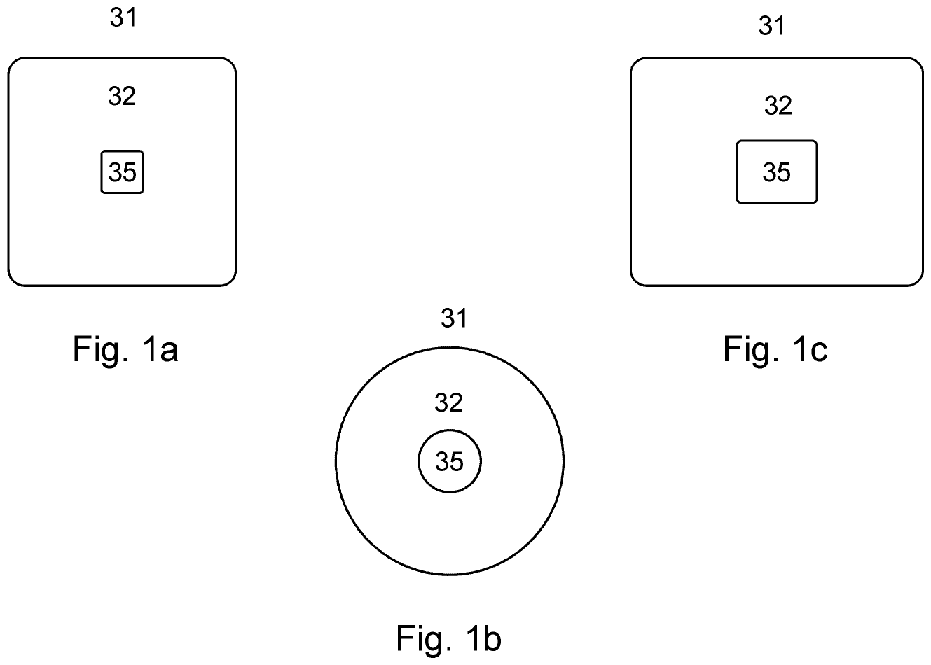

FIG. 1 a Top view of an in-situ stencil

FIG. 1 b Top view of another in-situ template

FIG. 1 c Top view of a third in-situ template

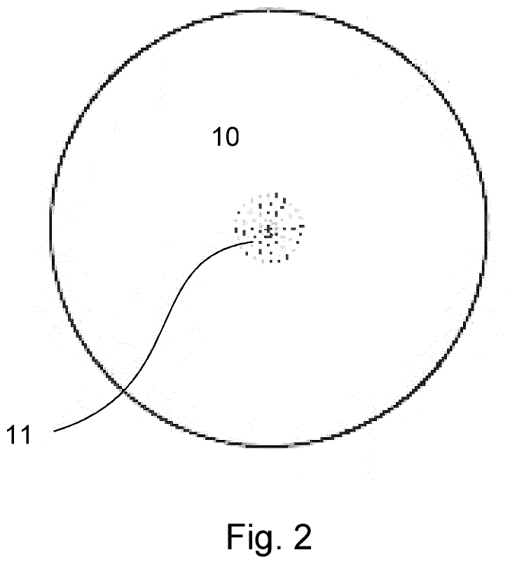

FIG. 2A top view of an embodiment of a rubber dam designed as a nipple protector

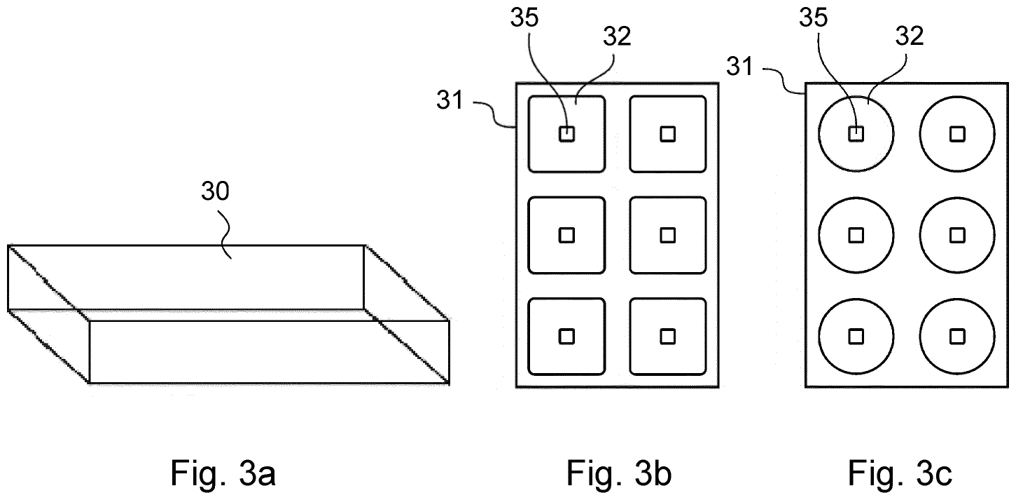

FIG. 3 a A cultivation vessel

FIG. 3 b In-situ template with six recesses

FIG. 3 c Further in-situ template with six recesses

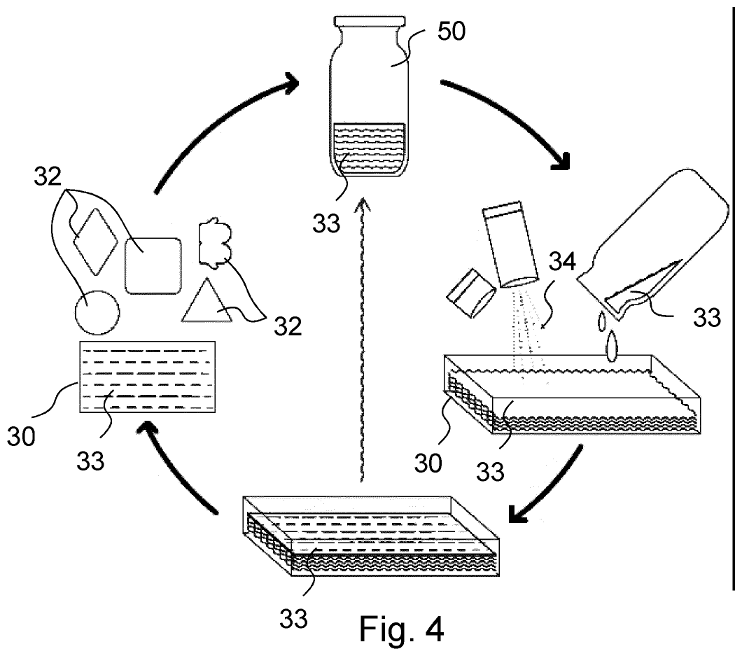

FIG. 4 An example of a process for the production of a rubber dam from bacterial nanocellulose

FIG. 1 shows different embodiments of in-situ templates 31 for producing a rubber dam 1. The templates 31 can have different shapes, here e.g. a square (FIG. 1 a), a circular (FIG. 1 b) or a rectangular (FIG. 1 c) shape. The templates 31 have the recess 32 in which the hydrogel is cultivated. The hydrogel is produced by microorganisms at the interface between air and a nutrient medium containing sugar. The bacteria expel the cellulose as fibrils, which aggregate to form fibers at the interface between the nutrient medium and air. The result is a three-dimensionally interwoven fiber network consisting of about 99% water and 1% nanocellulose. The area of the opening 35 or perforation 11, which corresponds to an opening in the manufactured rubber dam 1, is indicated in the center.

The templates 31 can also be punching or cutting molds 31. From a solidly grown block or a thin membrane of a hydrogel, shapes can be modeled with the aid of cutting and milling tools (knives, scissors, punches, lasers, perforated pipes) that would not be produced by purely organic growth. The nonwoven punched or cut from the hydrogel by means of the punching or cutting mold 31 has the same shape as a nonwoven cultivated from hydrogel by means of the corresponding template 31.

The template 31 can specify the final shape of a trunk 1 or individual components for a specific design of more complex models in a modular system. Using appropriate devices, cylinders, tubular covers, limbs and components can also be designed using the process described. The advantage over punching and cutting processes using knives, shears or lasers is the avoidance of waste and therefore efficient utilization of the raw materials used.

A rubber dam 1 according to the invention, here in the form of a nipple protector 10, is shown in FIG. 2. Nipple protectors 10 are used, for example, to protect sensitive areas of skin from overstressing, e.g. in competitive sports to reduce friction of the skin against clothing or—with a partial perforation 11 to care for the baby—for breastfeeding mothers.

A hydrogel with a diameter of 5-20 cm, a thickness of 0.5-3 mm and a central perforation 11 in a circular area with a diameter of up to 3 cm forms a functional protector that cushions and covers the nipple of breastfeeding mothers, while at the same time providing a natural haptic experience and care for the infant. The perforations can be made with needles or by laser cutting in various arrangements and degrees of permeability.

Washing with sodium hydroxide solution in a concentration and duration adapted to the strength and composition of the hydrogel makes the hydrogel more flexible, softer and more pliable in its cell structure without significantly reducing its stability and water retention capacity for its intended purpose. NaOH can be used in varying concentrations (0.1 molar to 1 molar) to inactivate the bacterial strains and to wash out endotoxins and other foreign substances from the culture medium during fermentation.

These principles of purification, boiling and sterilization by steam pressure or e-beam can now also be used to significantly extend the life cycle of the resistant hydrogels in their final product form and prepare them for reuse. Active ingredient-laden or contaminated hydrogels can thus be prepared for reuse on the skin and can be reused many times if used properly.

This allows deposits and unwanted bacteria to be washed out to ensure that the hydrogel is free of germs and impurities before it is used again. The pH value of a hydrogel can thus be influenced and adjusted to a desirable range for the skin.

FIG. 3 shows an example of a cultivation vessel 30 (FIG. 3 a) with suitably shaped in-situ templates 31 (FIG. 3 b, 3 c) for the production of a rubber dam 1 from bacterial nanocellulose. The cultivation vessel 30 is preferably made of glass or a food-safe plastic. The templates 31 correspond to the templates 31 from the first embodiment example (see FIG. 1) and are designed in this embodiment example in such a way that six rubber dams can be cultivated simultaneously in one cultivation vessel. The dimensions of the templates 31 are such that the templates 31 can be arranged parallel to the base surface of the cultivation vessel 30 in the cultivation vessel 30.

FIG. 4 shows an example of the production of a rubber dam 1. In particular, purified bacterial nanocellulose produced by various methods, such as the well-known Hestrin-Schramm method, based on coconut, acetic acid with Gluconacetobacter xylinus, cynobacteria (“blue-green algae”), as well as a variant from cannabis tea and Kombucha culture, is used here. The synthesis of bacterial nanocellulose can be organized with different raw materials, especially different carbon sources in the fermentation and thus be adapted to the local availability of resources.

Nanocellulose is preferably synthesized by cultivating microbial strains such as Gluconacetobacter, Enterobacter, Agrobacterium, Pseudomonas, and Rhizpobium. The bacteria are cultivated in the nutrient solution 33 contained in the cultivation vessel 30, supplemented if necessary with further active substances or nutrients 34 and can be stored in the container 50 for later inoculation of a culture medium. They can be mixed with nutrients and additives in water and thus initiate a new fermentation.

Alternatively or additionally, shapes can also be punched or milled from the thin membrane of the BNC nonwoven produced in this way with the aid of punching or cutting dies 31 using cutting and milling tools. The nonwoven then has a shape corresponding to the punched or cut shapes 31.

In addition to Gluctonacetobacter hansenii and Gluconacetobacter kombuchae, the bacterial strain Gluconacetobacter xylinus, which has also been most extensively researched and documented in this context, is particularly suitable for the production of dimensionally stable nanocellulose gels. The hydrogel is produced by microorganisms at the interface between air and a nutrient medium containing sugar. The bacteria expel the cellulose as fibrils, which assemble into fibers at the interface between the nutrient medium and the air. The result is a three-dimensionally interwoven fiber network consisting of approximately 99% water and 1% nanocellulose. The shape of the rubber dam 1 is determined by the template 31 and its openings 32.

In contrast to alkaline cleansing or loading by means of an inoculation substance 33 or with active ingredients 34 (such as aloe vera, hyaluronic acid, black tea), acidic loading with probiotic bacterial cultures may be desirable and give the hydrogel special properties, for example in the treatment of certain fungal diseases (with lactic acid and lactic acid bacteria), psoriasis or as a carrier of a specific bacterial culture for the cultivation of bacterial nanocellulose.

From a chemical point of view, bacterially synthesized nanocellulose (BNC) is identical to plant cellulose, but differs significantly due to the specific biosynthesis process. BNC typically has a fiber diameter of around 40-60 nm, which corresponds to one hundredth of the fiber diameter of a plant cellulose or cotton fiber. Depending on the manufacturing process, fiber diameters can also be in the range of 20-100 nm. The nonwovens and/or utensils produced according to the method of the invention have fibers with a diameter of 10 nm to 150 nm, preferably a diameter of 25 nm to 90 nm, particularly preferably a diameter of 50 to 70 nm. The fibers of the nonwovens and/or utensils produced according to the method of the invention have a length of 100 μm to 1 mm. Typically, the fiber length of the nonwoven is 5 μm to 15 mm. The density of the nonwoven is 1-1.5 g/cm³ and it has a tensile strength of 200-400 MPa.

The method of making the rubber dam further optionally includes the following steps: dissolving, in a flat-bottomed vessel, crystalline glucose having a concentration of from 2% to 20% by weight, sodium hydrogen phosphate and citric acid in water to form a buffered pH between pH 4 to pH 7, introducing a dry mixture of peptone and a yeast extract each having a concentration between 0.1% and 5% by weight into the buffered aqueous solution, stirring the solution until the peptone is completely dissolved and the yeast extract is dissolved. % and 5% by weight into the buffered, aqueous solution, stirring the solution until the peptone has completely dissolved and the yeast extract is fully suspended. Sterilization by autoclaving at 121° C. for 20 minutes. Inoculation with bacterial strain Gluconacetobacter xylinus, cultivation of the solution for between 2 days and 25 days until a hydrogel forms at the interface between the culture medium and the air, decanting of the aqueous solution, washing of the hydrogel, purification and subsequent optional soaking of the hydrogel in an aqueous solution containing colouring agents, flavourings, fragrances and active ingredients for between 30 minutes and 30 days, and finally sterilization by superheated steam or e-beam.

In an alternative method, instead of introducing glucose, peptone, yeast, sodium hydrogen phosphate and citric acid, the following step is carried out: Incorporating a powder comprising 2% to 10% by weight of an extract of black tea, green tea and/or cannabis tea, 90% to 98% by weight of sucrose and/or glucose, 1% to 5% by weight of fruit or vegetable powder, dried leaves and flowers and dried herbal flavors and active ingredients.

The inoculation is carried out by adding dried microbes or a liquid solution containing them, at least one species selected from the group comprising: Gluconacetobacter xylinus, Gluconacetobacter kombuchae, Komagataeibacter hansenii, Gluconobacter oxydans, Saccharomyces ludwigii, Saccharomyces apiculatus or Saccharomyces cerevisiae. In addition, at least one further organic acid with a concentration of 0.1% by weight to 5% by weight is added, selected from the group comprising: gluconic acid, glucuronic acid, dextrorotatory (L+) lactic acid, tartaric acid, folic acid, oxalic acid, usnic acid, succinic acid, malic acid, malonic acid and citric acid. The sum of the weight percentages of the ingredients together is 100% by weight.

Alternatively, instead of adding glucose, peptone, yeast, sodium hydrogen phosphate and citric acid, the following step can be carried out: Introduce a solution of 300 g of white refined beet sugar or cane sugar in 2 I of coconut water, as well as 120 ml of concentrated anhydrous acetic acid.

In a further embodiment of the method, instead of introducing glucose, peptone, yeast, sodium hydrogen phosphate and citric acid, and instead of inoculation with Komagataeibacter xylinus, the following steps are carried out: Introduction of a solution of 5 g of dried cannabis flowers or leaves boiled in 1000 ml of water with the addition of a teaspoon of coconut oil for 60 min, mixed with 100 g of sugar (white refined beet sugar or cane sugar), cooled to room temperature and introduction of 250 ml of acidic kombucha tea (pH 2.2-pH 3.5) containing an active kombucha culture (such as live Gluconacetobacter kombuchae).

A kit for the use of any of the aforementioned manufacturing methods comprises 2% to 10% by weight of an extract of black tea, green tea and/or cannabis, 90% to 98% by weight of sucrose and/or glucose, 1% to 5% by weight of fruit or vegetable powder, dried leaves and flowers, dried herbal flavors and active ingredients, and dried microbes of at least one species selected from the group consisting of: Gluconacetobacter xylinus, Gluconacetobacter kombuchae, Komagataeibacter hansenii, Gluconobacter oxydans, Saccharomyces ludwigii, Saccharomyces apiculatus or Saccharomyces cerevisiae.

The set also comprises at least one further organic acid with a concentration of 0.1 wt. % to 5 wt. % selected from the group comprising: acetic acid, gluconic acid, glucuronic acid, dextrorotatory (L+) lactic acid, tartaric acid, folic acid, oxalic acid, usnic acid, succinic acid, malic acid, malonic acid and citric acid. The sum of the weight percentages of the components together is 100% by weight. The set is composed in such a way that a pH value of 3.5 to 7 is formed in aqueous solution.

The process for producing bacterial nanocellulose with a dry instant mixture or the two-component solution described below is new. Key production parameters are standardized here, which leads to a greatly simplified and more predictable result. This process can also be used in the food sector (e.g. Kombucha drinks) or textiles (production of vegan leather or fabrics based on bacterial nanocellulose), as the process step of brewing and cooling the tea is no longer necessary and the mixing ratio between the ingredients remains constant. The instant blend is a great simplification, especially for home users.

Kombucha, with its traditional home brewing and fermentation culture, often appears as an undefined culture, but usually contains a desirable probiotic composition of bacteria and yeast strains. However, this can vary greatly due to the nature of wild fermentation. Known components included here are: Gluconoacetobacter xylinus, Gluconoacetobacter kombuchae, Gluconoacetobacter hansenii Acetobacter xylinoides, Gluconobacter oxydans, Saccharomyces ludwigii, Saccharomyces apiculatus, Saccharomyces cerevisiae.

Organic acids include acetic acid, gluconic acid, glucuronic acid, dextrorotatory (L+) lactic acid, tartaric acid, folic acid, oxalic acid, usnic acid, traces of succinic acid, malic acid, malonic acid and citric acid. Trace elements and minerals include iron, magnesium, sodium, potassium, calcium, copper, zinc, manganese, cobalt and other minerals.

The list of vitamins includes vitamin B1, vitamin B2, vitamin B3, vitamin B6, vitamin B12, vitamin C, vitamin D, vitamin E, vitamin K. It also contains various amino acids, enzymes, tannins, the enzymes invertase, amylase, catalase, saccharase, rennet ferment and a proteolytic ferment, antibiotic substances, alcohol and carbonic acid.

The bacterial cultures of kombucha have the special property of being able to assert themselves in sufficiently acidic liquid against foreign bacteria that threaten the system. If they are supplied with nutrients, natural colorants, active ingredients and flavors, the growing bacterial cellulose with its high water absorption (approx. 99% water, 1% cellulose) and its water retention capacity takes on additional properties.

Active nanocellulose gels contain live probiotic bacterial strains, in passive nanocellulose gels the bacterial strains were killed and extracted by purification process steps, and the hydrogels were sterilized by autoclaving (121° C. steam for 15-20 minutes) or electron beam process.

The combination of 250 ml of acidic Kombucha tea [“Fairment Kombucha-Original” pH 2.5-2.8] or a defined nanocellulose gel strain (pH 2.2-3.5) with a suitable active bacterial culture and 25 g of instant powder is suitable for producing one or more Kombucha-based nanocellulose gels with a total mass of 50 g or more in 2-25 days of standing cultivation under hygienic conditions and oxygen supply. The material properties of the kombucha-based nanocellulose gels are similar to the synthesized biopolymers mentioned above.

The liquid by-product is an acidic tea solution with the usual Kombucha composition of bacterial and yeast cultures (pH 2.3-4) with proportions of organic aroma, color, flavor and active ingredients (from fruit or vegetables, teas, plant aromas and active ingredients according to the composition of the instant powder). This solution is now suitable for inoculating and coloring, as well as for storing, renovating and maintaining a nanocellulose gel. It can be used to revitalize a sterilized, passive nonwoven with the probiotic culture.

Growth is also decisively influenced by the container. The shape, the surface, the filling level and the material are factors that determine the properties. Porcelain, plastic and glass containers are best suited for the cultivation of nanocellulose gels for home use.

In addition to synthesized, passive nanocellulose nonwovens and the probiotically active hydrogels, there is a third method of producing a nanocellulose gel based on coconut, which in turn has similar material properties to the above-mentioned nanocellulose gels and can also be loaded in situ or subsequently. Here, too, the production process of fermentation in stand cultivation over 2 days to 25 days is suitable, whereby the nutrient medium (pH 2.3-3.5) is composed as follows: 120 ml concentrated, anhydrous acetic acid (glacial acetic acid), 300 g sugar [Naturata organic beet sugar] (white, refined, alternatively raw cane sugar), 300 ml Nata starter (bacterial strain Gluconacetobacter xylinus), alternatively a Kombucha starter or non-pasteurized Kombucha drink [“Fairment Kombucha-Original”] can be used, also 2 I coconut water [“Coco Juice pure organic”].

A fourth way to produce bacterial nanocellulose is to use medicinally active cannabis. The nutrient solution is prepared by boiling 5 g of cannabis leaves or flowers with 1 I of water and a teaspoon of coconut oil for 60 minutes and adding 100 g of sugar [Naturata organic beet sugar] (white, refined, alternatively raw cane sugar). The addition of 250 ml of acidic Kombucha tea [Fairment Kombucha-Original pH 2.5-2.8] or a defined nanocellulose gel strain (pH 2.2-3.5) triggers the production of nanocellulose, which contains active cannabinoids in addition to the properties mentioned above. There are many receptors for cannabinoids in the skin and mucous membranes. The medicinally active ingredient cannabidiol (CBD), for example, promotes blood circulation in the tissue, which can lead to increased sensitivity.

The nanocellulose gels produced in different ways are preferably used in the following product variants: The wet wipe, rubber dam or protective film has a drained weight of 12-180 g with a size of 150-300 mm×150-300 mm and a thickness of 0.1-3 mm. The washcloth (or massage sponge or haptic stimulant) has a draining weight of 60-180 g for a size of 120-180 mm×120-180 mm and a thickness of 3-10 mm.

Both formats (with rounded corners or cut at right angles) can be combined with a glass or plastic cylinder, which can be filled with hot water or skin care products. A wash mitt results from the combination of two cloths (e.g. square, rectangular, in the shape of a hand, etc.), which can be cut, sewn and crimped or pressed in a similar way to textiles.

As a material unit for individual further processing, the nonwoven has a length of 150-400 mm and a width of 120-300 mm when not rolled. In rolled form, this results in a cylindrical shape of the stated length and, depending on the thickness of the material (0.5-25 mm), an outer diameter of 30-120 mm, with a drained weight of 100-1200 g. Sizes vary, as the products are both industrially manufactured and can be customized to meet the needs of individual consumers. By growing in a suitably shaped container-preferably made of glass or a food-safe plastic-foldable and rollable flaps (e.g. circular, oval, rectangular, square, diamond-shaped, triangular) can be produced with variable thickness/strength, depending on the duration of fermentation, the temperature and the addition of nutrients. Using appropriate devices and vessels, the process described can also be used to create various components such as cylinders, tubular coatings and protectors.

These nanocellulose gel components can be joined together using elastic bands, cords, cuffs, rings, clamps, clips or the technique of sewing. Massage tools, bags, containers such as jars, bottles or tubes can be combined in a shaping and stabilizing way or used for storage.

Tools such as templates made of plastic, glass or cork can also be used in the rearing process to shape the bacterial nanocellulose gel growing on the surface while it is still growing. This can represent the final shape or individual components for a specific design of more complex nanocellulose gel-based models. Lasers, embossing dies, stamping tools and branding dies can be used to mark or design objects made of bacterial nanocellulose with model numbers, manufacturing data or other information.

The basic reusability of the materials used, the many possibilities for up- and downcycling (e.g. use on the skin, later use as an antiviral disinfectant wipe or cleaning cloth, or in hydroponics and irrigation of plants) and thus a comparatively long product life cycle result in great potential for reducing packaging waste and disposable products. In the case of disposal, the dry mass is only about 1% of the hydrogenated product form and remains-depending on the previous loading-biocompatible, organic cellulose, which is thermally decomposed at 350° C.

The ideal duration of purification following cultivation and the concentration of NaOH (in distilled water) depends on the exact composition of nutrients and bacteria used, whether they are composites, hybrids or pure cultures.

In one embodiment of the process according to the invention, a one-step cleaning process is used. For each millimeter of thickness of the nonwoven, a reliable termination of all bacterial activity is achieved with a reaction time of 2 hours at 85° C. in 100 ml of 0.8 wt. % NaOH solution per cubic centimeter of cellulose in all the processes mentioned. Depending on the desired degree of purity, this process step can be repeated with the renewal of the sodium hydroxide solution as often as necessary- or in a dynamic cycle-until this NaOH solution absorbs no or only minor detectable impurities from the cellulose produced. The nonwoven is then rinsed with distilled water and, depending on the intended use, the pH is adjusted to the desired value between pH 4 and pH 7-preferably to a skin-neutral value of about 7, so that the pH can be easily readjusted by subsequent appropriate loading. Optionally, citric acid can also be used in addition to distilled water for this neutralization step.

The purified nonwoven has an (intended) loading of 8% by weight and 1.5% by weight of impurities. Purification can optionally be completed with sterilization, for example by hot steam for 20 minutes at 121° C. or e-beam, in order to kill other microorganisms and ensure the longest possible shelf life in the packaging.

In order to ensure the best possible shelf life for the trade, the packaging should ideally be vacuum-packed in water-impermeable film, without air supply in liquid (distilled water or in loading solution) and in terms of sustainability in a reusable, sealable cylindrical container or the bacterial nanocellulose freeze-dried in water-repellent or waterproof packaging for subsequent swelling.

In a further embodiment of a one-step cleaning process, the nonwoven is cleaned at 110° C. for 240 minutes with a 45% by weight NaOH solution. The amount of NaOH solution is also 100 ml per cubic centimeter of cellulose. After cleaning, the nonwoven is also rinsed with distilled water and optionally citric acid. The nonwoven cleaned in this way has a loading of 9.5% by weight and 0.8% by weight of impurities.

In a further embodiment of the method according to the invention, a two-stage cleaning process is used. In the first stage (coarse cleaning), a NaOH solution with 50 wt. % NaOH is used, the duration of action is 135 min, the temperature 127° C. In the second cleaning stage, the nonwoven is cleaned with an 8% by weight NaOH solution at a temperature of 85° C. for 240 minutes. The web cleaned in this way has a loading of 10.5% by weight and 0.27% by weight of impurities.

### LIST OF REFERENCE SYMBOLS

1 Utensil/Kofferdam
10 Nipple shield
11 Perforation
30 Cultivation vessel
31 In-situ stencil
32 Base/recess of an in-situ template for cultivation
33 Nutrient solution inoculated with bacterial culture
34 Nutrients/additives
35 Survey
50 Screw-top jar

1. Process for reprocessing a utensil (1) made of bacterial nanocellulose loaded with an additive, comprising the following process steps:
Washing the loaded utensil (1) in a lye solution
Sterilize the utensil (1).

2. Process for reprocessing a utensil (1) made of bacterial nanocellulose loaded with an additive according to claim 1
characterized in that
the hydrogel resulting from the cultivation is washed in an alkaline solution containing 5 wt. % to 50 wt. % at 37° C. to 142° C. for 5 min to 400 min.

3. Process for reprocessing a utensil (1) made of bacterial nanocellulose loaded with an additive according to claim 2
characterized in that
the lye is a 5 wt. % to 50 wt. % sodium hydroxide solution.

4. Method for reprocessing a utensil (1) made of bacterial nanocellulose loaded with an additive according to claim 1
characterized in that
a relative movement is generated between the washing solution and the hydrogel during the washing process.

5. Method for reprocessing a utensil (1) made of bacterial nanocellulose loaded with an additive according to claim 1
characterized in that
the washing process is carried out in two stages.

6. Process for reprocessing a utensil (1) made of bacterial nanocellulose loaded with an additive according to claim 5
characterized in that
the washing solution is renewed between the first and second stage of the washing process.

7. Method for reprocessing a utensil (1) made of bacterial nanocellulose loaded with an additive according to claim 1
characterized in that
sterilization is carried out after the washing process.

8. Process for reprocessing a utensil (1) made of bacterial nanocellulose loaded with an additive according to claim 7
characterized in that
sterilization is performed with steam or e-beam.

9. Method for reprocessing a utensil (1) made of bacterial nanocellulose loaded with an additive according to claim 1
characterized in that
the utensil (1) is only partially made of bacterial nanocellulose loaded with an additive.

10. Rubber dam (1) made from a hydrogel
characterized in that
the rubber dam (1) is made from a hydrogel of bacterial nanocellulose.

11. Rubber dam (1) made of a hydrogel according to claim 10
characterized in that
the rubber dam is made of bacterial nanocellulose with a cellulose content of more than 0.5% by weight.

12. Rubber dam (1) made of a hydrogel according to claim 10
characterized in that
the hydrogel made of bacterial nanocellulose is loaded with water and another substance.

13. Rubber dam (1) made of a hydrogel according to claim 12
characterized in that
the other substance is a pharmaceutical

14. Rubber dam (1) made of a hydrogel according to claim 10
characterized in that
the rubber dam (1) has a water content of between 7.5% and 99.5% by weight.

15. Rubber dam made of a hydrogel according to claim 10
characterized in that
the rubber dam (1) has a thickness of between 0.1-3 mm.

16. Rubber dam (1) made of a hydrogel of bacterial nanocellulose according to claim 10
characterized in that
the rubber dam (1) is reprocessed by a method comprising
washing the loaded utensil (1) in a lye solution; and
sterilizing the utensil (1).

17. Use of a hydrogel made of bacterial nanocellulose as a rubber dam (1).
